(19) United States
(12) Patent Application Publication
(10) Pub. No.: US 2009/0192580 A1
(43) Pub. Date: Jul. 30, 2009

(54) MEDICAL ELECTRICAL LEAD WITH BIOCOMPATIBLE LEAD BODY COATING

(76) Inventor: Shrojalkumar Desai, Little Canada, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/250,890

(22) Filed: Oct. 14, 2008

Related U.S. Application Data
(60) Provisional application No. 61/024,058, filed on Jan. 28, 2008.

(51) Int. Cl. A61N 1/05

(52) U.S. Cl. 607/127; 607/116

(57) ABSTRACT

An intravascular medical electrical lead includes a conformal coating including at least one layer formed from a poly-p-xylylene polymer. The coating is lubricious and provides an effective barrier against moisture and gases preventing degradation of the lead body material preventing corrosion of the conductor.

### TECHNICAL FIELD

The present invention relates to medical electrical leads. More particularly, the present invention relates medical electrical leads having a biocompatible coating.

### BACKGROUND

Implantable medical devices for treating irregular contractions of the heart with electrical stimuli are well known. Exemplary examples of such devices, which include defibrillators and pacemakers, generally include a medical electrical lead for delivering pacing therapy to the heart connected to a pulse generator. Such leads generally have an elongated, flexible insulating body, one or more inner conductors extending through lumens formed in the body and one or more exposed electrodes connected to the distal ends of the conductors. Important characteristics of the medical electrical leads include biocompatibility, durability, and reduced diameter.

When inserted within a patient's body, the lead body becomes subjected to the harsh body environment. Eventually, the lead body insulation begins to break down as a result of oxidation of the thermoplastic polymer or copolymer used to form the insulation. Oxidation of the polymer forming the insulation weakens the overall insulation. This may lead to cracks in the lead body insulation which can cause a breach potentially allowing bodily fluids to enter the lead and form a short between the conductor and/or the pulse generator generating current through the conductor. Thus, one challenge in lead body construction has been to prevent the breakdown of the lead body insulation caused by oxidation when subjected to the body environment.

### SUMMARY

According to one embodiment of the present invention, an intravascular medical electrical lead includes an insulative lead body having an outer surface extending from a proximal end adapted to be connected to a pulse generator to a distal end. According to various embodiments, an outer diameter of the lead body ranges from about 2 to about 15 French. At least one electrode is coupled to at least one conductor contained within the lead body. The lead body can include a single lumen or can include multiple lumens.

In various embodiments of the present invention, the intravascular medical electrical lead includes a parylene coating provided over an outer and/or an inner surface of at least a portion of the lead body. In further embodiments, the parylene coating is provided over the outer surface of the insulative lead body such that the coating coats the lead body from substantially the proximal end to the distal end of the lead body. According to other embodiments, the parylene coating is disposed on an inner surface of one or more of the lead's lumens.

According to various embodiments, the conformal coating is substantially pin-hole free and has a thickness ranging from about 0.1 μm to about 100 μm. In other embodiments, the conformal coating is substantially pin-hole free and has a thickness ranging from about 0.5 μm to about 25 μm.

According to further embodiments of the present invention, the parylene coating includes at least one layer including a poly-p-xylylene based polymer. In one further embodiment, the poly-p-xylylene polymer is Parylene N. In another further embodiment, the poly-p-xylylene polymer is Parylene C, Parylene D, or Parylene HT®. According to yet further embodiments of the present invention, the poly-p-xylylene polymer is an FDA approved poly-p-xylylene polymer. According to still further embodiments, the parylene coating can include co-polymers of a poly-p-xylylene polymer.

According to various embodiments of the present invention, an intravascular medical electrical lead includes a coating comprising a polymer having a structural repeating unit of

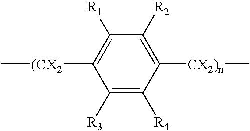

Wherein X is hydrogen or a halogen, R₁, R₂, R₃, and R₄ are each independently hydrogen, a halogen, an alkyl group, an alkyl halide, amino, nitro, alkylamine, alkyl hydroxy, or an alkyl carboxy group and n is at least 2. In various embodiments of the present invention, the coating is provided over an outer surface of at least a portion of an insulative lead body. In various other embodiments of the present invention, the coating is provided over an outer surface of an insulative lead body such that it coats the lead body from substantially the proximal end to the distal end. In some embodiments, the parylene coating is disposed over at least a portion of the inner surface of one or more lumens formed in the lead body.

In a further embodiment of the present invention, the polymer has a static coefficient of friction of less than about 0.40.

In another further embodiment, the polymer has a dielectric constant of less than about 3.25 at a frequency of 60 Hz.

In yet another further embodiment, the polymer has a moisture vapor transmission rate of less than about 1.7 g-mil/100 in²-24 hr at 37° C. and 90% Relative Humidity.

According to another further embodiment, the coating is substantially pin-hole free and has a thickness ranging from about 0.1 μm to about 100 μm.

According to yet another further embodiment, the coating is substantially pin-hole free and has a thickness ranging from about 0.5 μm to about 25 μm.

### BRIEF DESCRIPTION OF THE DRAWINGS

FIG. 1 is a schematic view of a medical electrical lead according to an embodiment of the present invention.

FIG. 2A is side cross-sectional view of a portion of a lead body according to an embodiment of the present invention.

FIG. 2B is an end, cross-sectional view of the lead body shown in FIG. 2A according to an embodiment of the present invention.

FIG. 3 is an end, cross-sectional view of a lead body according to an embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

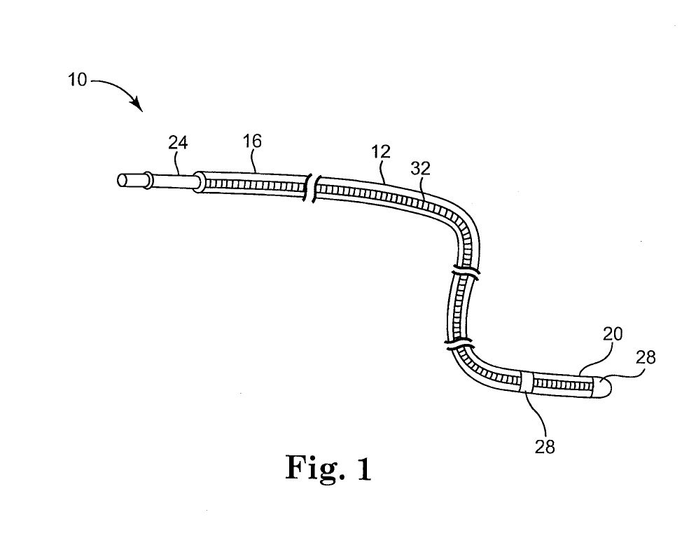

FIG. 1 is a partial cross-sectional view of a medical electrical lead 10, according to an embodiment of the present invention. Medical electrical lead 10 includes an elongated, insulative lead body 12 extending from a proximal end 16 to a distal end 20. The proximal end 16 is configured to be operatively connected to a pulse generator via a connector 24. At least one conductor 32 extends from the connector 24 at the proximal end 16 of the lead 10 to one or more electrodes 28 at the distal end 20 of the lead 10. The conductor 32 can be a coiled or cable conductor. According to some embodiments where multiple conductors are employed, the lead can include a combination of coiled and cable conductors. When a coiled conductor is employed, according to some embodiments, the conductor can have either a co-radial or a co-axial configuration.

The lead body 12 is flexible, but substantially non-compressible along its length, and has a circular cross-section. According to one embodiment of the present invention, an outer diameter of the lead body 12 ranges from about 2 to about 15 French. The medical electrical lead 10 may be unipolar, bipolar, or multi-polar depending upon the type of therapy to be delivered. In embodiments of the present invention employing multiple electrodes 28 and multiple conductors 32, each conductor 32 is adapted to be connected to an individual electrode 28 in a one-to-one manner allowing each electrode 28 to be individually addressable. Additionally, the lead body 12 can include one or more lumens. In some embodiments at least one lumen is adapted to receive the insertion of a conductor during construction of the medical electrical lead. In further embodiments, at least one lumen is adapted to receive a guiding element such as a guidewire or a stylet for delivery of the lead 10 to a target location within a patient's heart.

The electrodes 28 can have any electrode configuration as is known in the art. According to one embodiment of the present invention, at least one electrode can be a ring or partial ring electrode. According to another embodiment, at least one electrode 52 is a shocking coil. According to yet another embodiment of the present invention, at least one electrode 28 includes an exposed electrode portion and an insulated electrode portion. In some embodiments, a combination of electrode configurations may be used. The electrodes 28 can be coated with or formed from platinum, stainless steel, MP35N, a platinum-iridium alloy, or another similar conductive material. In further embodiments, a steroid eluting collar may be located adjacent to at least one electrode 28.

According to various embodiments, the lead body 12 can include one or more fixation members for securing and stabilizing the lead body 12 including the one or more electrodes 28 at a target site within a patient's body. The fixation member(s) can be active or passive. In some embodiments, the fixation member can be a screw-in fixation member. In other embodiments, the fixation member can be an extendable/retractable fixation member and can include one or more mechanical components adapted to facilitate the extension/retraction of the fixation member. An exemplary extendable/retractable fixation member is shown and described in U.S. Pat. No. 6,463,334 which is herein incorporated by reference.

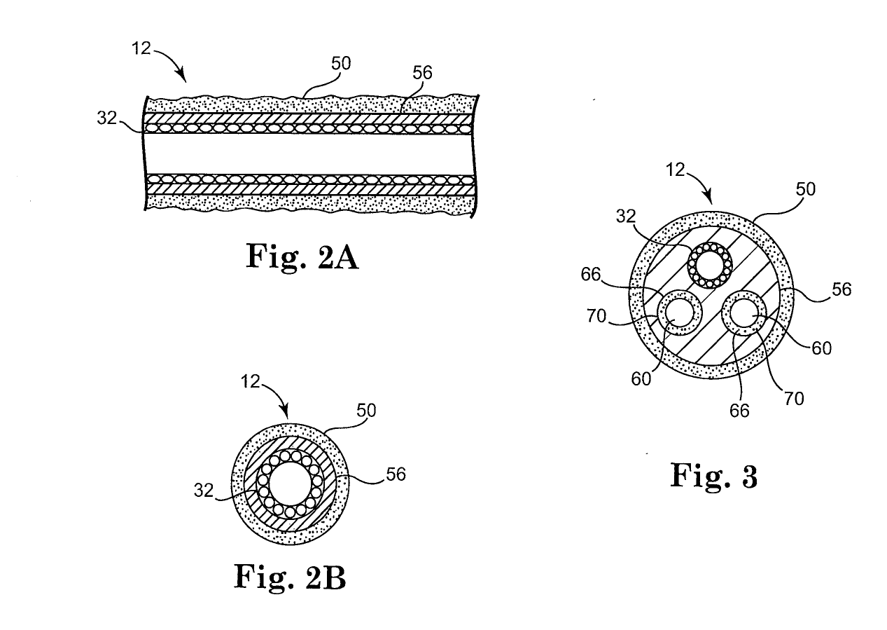

FIGS. 2A and 2B are cross-sectional views of a portion of a lead body 12, according to various embodiments of the present invention. As shown in FIGS. 2A and 2B, the insulative lead body 12 includes a coating 50 formed from parylene or a derivative thereof provided over at least a portion of an outer surface 56 of the insulative lead body 12. According to some embodiments of the present invention, the coating 50 is provided over the outer surface of the insulative lead body such that the coating 50 extends from substantially the proximal end to the distal end of the lead body 12. According to other embodiments of the present invention, the coating 50 is provided at one or more discrete locations along the lead body 12. The coating 50 is provided at one or more locations located on the lead body 12 that are subject to additional mechanical or physical stresses when implanted in a patient's body. The parylene coating provides an additional barrier or reinforcement against degradation and/or deterioration of the lead body 12 resulting from the additional mechanical and/or physical stresses placed on those particular locations.

FIG. 3 is a cross-sectional view of a lead body 12 including multiple lumens 60 according to various other embodiments of the present invention. As shown in FIG. 3, a parylene coating 66 is disposed over the inner surface 70 of one or more of the lumens 60. In some embodiments, the parylene coating 66 can be disposed over at least a portion of the inner surface 70 of the one or more lumens located in the lead body 12. In other embodiments, the parylene coating 66 may be disposed over the inner surface 70 of the one or more lumens from approximately the proximal end to the distal end of the lead body.

According to various embodiments of the present invention, a lead body 12 may include a parylene coating disposed over the outer surface of the lead body 12 and a parylene coating disposed over the inner surface of one or more lumens 60 located in the lead body.

According to yet further embodiments of the present invention, a parylene coating may be provided over the extension/retraction mechanism of an extendable/retractable fixation member facilitating its operation and/or the inner surface of the lumen or cavity in which the mechanism and fixation member is disposed.

“Parylene” is a generic name used to describe a class of poly-p-xylylenes. Poly-p-xylylene polymers and derivatives thereof typically have a repeating structure of

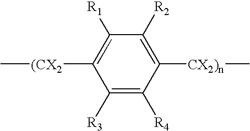

wherein X is a halogen or a hydrogen, and R₁, R₂, R₃, R₄ are each independently a hydrogen, a halogen, an alkyl, an alkyl halide, an alkyl halide, amino, nitro, alkylamine, alkyl hydroxy, or an alkyl carboxy group and n is at least 2. Two commercially available forms of parylene include Parylene N and Parylene C. Parylene N is poly-para-xylylene and has the following repeating structural unit shown below:

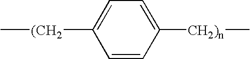

Parylene N has a high dielectric strength and provides a dielectric constant that is independent of frequency. Parylene N is adapted to be used at temperatures exceeding 220° C. Parylene C is poly-monochloro-para-xylene, and has the following repeating structural unit shown below:

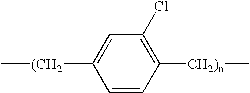

Parylene C provides a combination of physical and electrical properties including low permeability to moisture and corrosive gases. Both Parylene N and Parylene C comply with the United States Pharmacopeia's (USP) Class IV biological testing requirements and are approved for use in medical applications by the Food and Drug Administration (FDA). A third form of parylene, Parylene D, is also available. Parylene D exhibits greater thermal stability than Parylene N or Parylene C. Parylene D is poly-dichloro-para-xylene has the following repeating structural unit shown below.

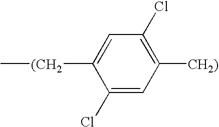

Fluorinated poly xylylene based polymers can also be used. An exemplary fluorinated poly xylylene based polymer includes Parylene HT®, also known as Parylene F. Parylene HT® is commercially available from Specialty Coating Systems located on the World Wide Web at www.scscoatings.com. Parylene HT® has the repeating structural unit shown below.

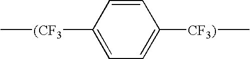

Parylene HT® has a lower dielectric constant than the other parylene variants and offers greater thermal stability. The lower dielectric constant coupled with the higher thermal stability may make Parylene HT® useful in MRI compatible applications. Additionally, Parylene HT® has a low coefficient of friction (dynamic and static) making it useful as a lubricious coating.

A table listing some of the properties of Parylene N, Parylene C, Parylene D, and Parylene HT® is provided in Table 1.

TABLE 1

| | Parylene | Parylene | Parylene | Parylene |
|---|---|---|---|---|
| | N | C | D | HT ® |
| Tensile Strength, psi | 6,500 | 10,000 | 11,000 | 7,500 |
| Dielectric Strength, | 7,000 | 6,800 | 5,500 | 5,600 |
| short time | | | | |
| (Volts/mil at 1 mil) | | | | |
| Dielectric Constant: | 2.65 | 3.15 | 2.84 | 2.2 |
| 60 Hz | | | | |
| Coefficient of Friction: | 0.25 | 0.29 | 0.33 | 0.14 |
| static | | | | |
| Coefficient of Friction: | 0.25 | 0.29 | 0.31 | 0.13 |
| Dynamic | | | | |
| Gas Permeability*: | | | | |
| Nitrogen | 7.7 | .095 | 4.5 | — |
| Oxygen | 30 | 7.1 | 32 | 23.5 |
| Carbon Dioxide | 214 | 7.7 | 13 | — |
| Hydrogen Sulfide | 795 | 13 | 1.45 | — |
| Sulphur Dioxide | 1,890 | 11 | 4.75 | — |
| Chlorine | 74 | 0.35 | 0.55 | — |
| Moisture Vapor | 1.50 | 0.14 | 0.25 | <0.1 |
| Transmission Rate** | | | | |
| Water Absorption (%) | <0.1 | <0.1 | — | <0.1 |

*cm³-mil/100 in²-24 hr-atm (23° C.)
**g-mil/100 in²-24 hr, 37° C., 90% RH
1 mil = 1/1000 in. = 25.4 microns

According to various embodiments of the present invention the parylene coating may include one or more derivatives of parylene, parylene derivatives and/or co-polymers thereof. According to other various embodiments the parylene coating may be applied in one or more layers. The layers need not include the same parylene compound with each subsequent layer. Additionally, layers of coating including parylene may be alternated with layers of coating including other suitable insulative material such as polyurethane and co-polymers thereof.

The parylene coating can be applied in a very thin layer to the outer surface of the insulative lead body such that it does not add to the overall weight or the outer diameter of the lead body, but yet still provides an effective barrier against moisture and gases preventing degradation of the lead body material and protecting the conductor contained within the lead body 12 from corrosion. A parylene coating offers a lubricity comparable to that of Teflon® because of its low static and dynamic coefficients of friction. As such, parylene can be used to replace conventional materials such as poly tetrafluoroethylene and used to construct lead bodies having reduced outer diameters.

According to various embodiments of the present invention, as described above, the parylene coating is non-porous and substantially pin hole free and conforms to the outer and or inner surfaces the insulative lead body 12. A non-porous, pin hole free parylene coating may act as a barrier, protecting the lead body from the harsh environment within the body, thus protecting the insulative lead body 12 from degradation and/or deterioration. Degradation and/or deterioration of the lead body 12 can result from the breakdown of the polymer used to form the lead body 12. Breakdown of the polymeric material used to form the lead body 12 can make the lead body 12 more susceptible to physical, chemical, and mechanical stresses resulting from implantation in a patient's body.

A parylene coating provided over the outer surface of the lead body and/or the inner surface of one or more of the lead body's lumens also may improve the MRI compatibility of the lead. The parylene coating provides a low thermally conductive barrier over the lead body shielding the conductor(s) located inside. This may prevent the conductor(s) from heating or transferring heat to the surrounding tissue in response to the electromagnetic radio frequency waves generated during MRI imaging. Additionally, the parylene coating(s) may shield the conductor from the radio frequency waves, preventing an inducting of current in the conductor.

In addition to being a conformal surface coating, a parylene coating is also a lubricious coating. When provided over an outer surface of the lead body, a lubricious coating including parylene may aid in the insertion and location of the lead at a target therapy site within a patient's body. When provided over the inner surface of a lumen, the parylene coating may aid in the lead body construction by facilitating the insertion of one or more conductors into the lead body. A parylene coating provided over the extension/retraction mechanism or the surfaces of the lumen or cavity in which it is disposed may act as both a lubricant facilitating operation of the fixation mechanism as well an anti-coagulant or barrier to any blood that may seep into the mechanism that would otherwise create friction decreasing the operability of the mechanism.

According to various embodiments of the present invention, the parylene coating can be deposited on a substrate (e.g., an insulative lead body) by vapor deposition polymerization techniques known to those of skill in the art. An exemplary method of depositing parylene or a parylene derivative on a substrate by vapor deposition polymerization is shown and described in U.S. Pat. No. 5,424,097, which is incorporated herein by reference. According to other embodiments of the present invention, a conformal coating formed from parylene or a parylene derivative can be deposited on a substrate by plasma vapor deposition techniques known to those of skill in the art. In some embodiments, the substrate can be pre-treated to facilitate the deposition of the parylene coating. Surface pre-treatment can be performed by a number of techniques including plasma and reactive gas techniques known to those of skill in the art.

Vapor deposition polymerization of parylene begins with a powdered form of a parylene dimmer, since the parylene monomer is not stable. Sublimated directly to a vapor and cracked to a monomeric state, the resultant parylene coating forms by spontaneous polymerization on the target substrate, such as the outer surface of the insulative lead body 12, in an evacuated, room-temperature deposition chamber. The parylene coating grows from the monomeric vapor onto the surface of the substrate one molecule at a time, facilitating the formation of a conformal, uniform coating on the substrate.

Vapor deposition polymerization facilitates the formation of a thin, conformal coating having a uniform thickness that is substantially non-porous and free from pinholes. Vapor deposition polymerization also facilitates the precise control of coating thickness. According to one embodiment of the present invention a parylene coating 50 has a thickness ranging from about 0.1 μm to about 100 μm. According to other embodiments, a parylene coating has thickness ranging from about 0.5 μm to about 95 μm. According to yet another embodiment of the present invention, the parylene coating has a thickness ranging from about 0.5 μm to about 5 μm.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. An intravascular medical electrical lead comprising:
an insulative lead body having an outer surface and at least one lumen having an inner surface, the lead body extending from a proximal end adapted to be connected to a pulse generator to a distal end;
at least one conductor contained within the insulative lead body;
at least one electrode operatively coupled to the at least one conductor; and
a parylene coating provided over at least a portion of the outer surface of the insulative lead body, wherein the conformal coating is substantially pin-hole free and has a thickness ranging from about 0.1 μm to about 100 μm.

2. The medical electrical lead according to claim 1, wherein the parylene coating comprises Parylene N.

3. The medical electrical lead according to claim 1, wherein the parylene coating comprises Parylene C.

4. The medical electrical lead according to claim 1, wherein the parylene coating comprises Parylene D.

5. The medical electrical lead according to claim 1, wherein the parylene coating comprises Parylene HT®.

6. The intravascular medical electrical lead according to claim 1, wherein the coating is substantially pin-hole free and has a thickness ranging from about 0.5 μm to about 5 μm.

7. The medical electrical lead according to claim 1, wherein the thickness of the coating ranges from about 0.5 μm to about 95 μm.

8. The intravascular medical electrical lead according to claim 1, wherein the polymer has a static coefficient of friction of less than about 0.40.

9. The intravascular medical electrical lead according to claim 1, wherein the polymer has a dielectric constant of less than about 3.2 at a frequency of 60 Hz.

10. The intravascular medical electrical lead according to claim 1, wherein the parylene coating coats the lead body from substantially the proximal end to the distal end of the lead body.

11. The intravascular medical electrical lead according to claim 1, wherein the parylene coating coats the inner surface of the at least one lumen of the lead body.
